(19) United States
(12) Patent Application Publication
(10) Pub. No.: US 2009/0239943 A1
(43) Pub. Date: Sep. 24, 2009

(54) Nutraceutical composition and methods for its use

(76) Inventor: Fazlul H. Sarkar, Plymouth, MI (US)

Correspondence Address: Hoffman, Wasson & Gitler, P.C., Suite 522, 2461 South Clark Street, Arlington, VA, 22202, US

(21) Appl. No.: 12/382,520

(22) Filed: Mar. 18, 2009

Related U.S. Application Data
(60) Provisional application No. 61/037,734, filed on Mar. 19, 2008.

(51) Int. Cl. A61K 31/352; A61P 29/00

(52) U.S. Cl. 514/456

(57) ABSTRACT

A composition for inhibiting the activity of NF kappa B or for inhibiting tissue damage inflammation due to disease including a combination of green tea polyphenols; resveratrol; soy isoflavones, and curcumin. Also disclosed is a method of inhibiting the activity at the NF kappa B or inflammation using the composition.

### CROSS-REFERENCES TO RELATED APPLICATION

This application claims the benefit of U.S. Provisional Patent Application Ser. No. 61/037,734, filed Mar. 19, 2008.

### FIELD OF THE INVENTION

This invention relates generally to nutraceutical compositions and to their use, either singly or in combination with other treatments and materials, to promote health or treat disease conditions. More particularly the invention relates to a particular combination of naturally occurring herbal materials and/or extracts therefrom which interact synergistically to provide an enhanced effect on the promotion of health, the prevention of sickness and/or the treatment of diseases.

### BACKGROUND OF THE INVENTION

General systemic and organ specific inflammation and tissue damage, both acute and chronic, is associated with a number of disease conditions. Such tissue damage and inflammation is caused by a number of factors which can be external to the organism and/or generated internally. These factors can include free radicals and other chemical agents which can damage tissue, provoke immune responses, or cause mechanical damage and the like. In a large number of instances, inflammatory processes involve the induction of expression and/or the activation of a particular protein known as nuclear transcription factor kappa B (NF kappa B). For example, in experimental models for the development of several cancers, agents which promote tumor growth such as TNF have been found to activate NF kappa B.

It has been found that NF kappa B activity increases in response to both inflammatory and oxidative signals through multiple pathways and plays a role in the promotion, exacerbation and/or prolongation of various disease conditions. It has been found that inhibition of the activity of NF kappa B can have beneficial effects on health, wellbeing and the progression and treatment of various diseases.

Various chemical agents are known to inhibit or moderate the activation of NF kappa B. These agents include a number of compounds known to have antioxidant effects. For example, curcumin, which is derived from turmeric, has been shown to have an effect in inhibiting NF kappa B, and as such is of value in the treatment of a number of disease conditions. However, curcumin, like may other therapeutic agents, acts upon a number of discrete chemical pathways active in different parts of a cell or organism. The action of such compounds inhibits particular pathways while potentiating others. As a result, such agents often manifest a combination of beneficial and detrimental effects. For example, some compounds which inhibit particular biochemical pathways associated with the progression of cancer can also promote particular cancers, either by stimulating certain pathways or inhibiting other pathways. The same combination of effects also is manifested by other compounds, herbal extracts and methods. For example, antitumor agents effective in one tissue may promote tumor growth in another. Likewise, therapies such as radiation or surgery can produce mechanical or oxidative stress in tissues which will lead to adverse effects.

As will be detailed hereinbelow, the present invention is directed to a synergistic combination of materials which are individually effective as agents for promoting healing, wellbeing and general health of tissues. The particular combination of agents interacts synergistically so as to provide a maximum beneficial effect while mitigating adverse effects of other members of the combination. The composition may be used alone for the promotion of health and the prevention of disease; with other health promoting agents, such as vitamins; or in conjunction with therapies for disease including chemotherapies, surgery, radiation, thermal therapies and the like.

### DESCRIPTION OF THE INVENTION

The present invention comprises a synergistic combination of agents active to mitigate the activity of NF kappa B and/or other pathways, and/or otherwise decrease the effects of oxidative or inflammatory damage to tissues. The combination of agents of the present invention may be used alone as a therapeutic material for promoting health, wellbeing and treating diseases. However, the composition may also be used, with advantage, in combination with further health promoting or therapeutic materials and methods. For example, the materials and methods of this invention may be used in combination with nutraceutical materials for the promotion of health and wellbeing; they may also be used in conjunction with medical methods and materials in various therapies. In cancer therapies, the composition of the present invention may be administered in combination with conventional chemotherapeutic agents as well as with surgery, radiation or thermal therapies; and, the composition of the present invention operates to protect tissues from inflammatory or oxidative processes which can inhibit the effectiveness of other health promoting agents or further therapeutic methods or materials. The compositions of the present invention may be administered prior to, along with, or following such other health promoting agents or therapeutic materials and methods.

The compositions of the present invention are based upon a combination of polyphenols such as green tea polyphenols, together with resveratrol, isoflavones, and curcumin.

Green tea polyphenols comprise a mixture of polyphenolic compounds extracted from tea leaves. These compounds have been shown to be good scavengers for free radicals as well as for other activated chemical species such as activated carbonyls and the like. Green tea polyphenols are available from a number of sources and are generally prepared by extraction processes involving water and alcohols, typically ethanol. One particular material having use in the present invention is available from Amax NutraSource, Inc. under the designation green tea 50% polyphenols. Like materials from other suppliers may be utilized in this invention.

Resveratrol is another natural product having strong anti-inflammatory and antioxidant properties. Resveratrol may be obtained from a number of sources including grapes as well as other botanicals. One particular material used in the present invention comprises a product sold by Amax NutraSource, Inc. under the designation resveratrol 20% and this material comprises an extract of the plant Polygonum cuspidatum prepared by an ethanol/water extraction process. The material as supplied comprises at least 20% resveratrol.

The isoflavone component of the present invention also is known to exert a number of biological effects. The isoflavone material is typically prepared by extraction from soybeans (Glycine max), although similar compounds may be obtained from other plant sources. One isoflavone material which may be used in the present invention comprises the product sold under the designation soy isoflavones 40% by Amax NutraSource, Inc. This material comprises an ethanol/water extract of soybeans and comprises approximately 40% total isoflavones, which comprise a mixture of compounds that include daidzin, daidzein, genistin, genistein, glycitin, and glycitein, among others. The isoflavone component may comprise a mixture of all, or some, of the foregoing isoflavones; or, it may comprise only one of the isoflavones.

The fourth component of the composition comprises curcumin. As is known in the art, curcumin is a polyphenolic diketone obtained from turmeric root. It has a number of anti-inflammatory actions. One source of the curcumin used in the present invention comprises turmeric powder available from Amax NutraSource, Inc. under the designation turmeric 95%.

The foregoing materials may be used in combination with yet other compounds, such as antioxidant vitamins or minerals that are known to be involved in stimulating the antioxidant activity of certain enzymes, as will be apparent to those of skill in the art. Likewise, various compositional ranges of the components may be employed. Compositions may include three of the foregoing materials, while in other instances they will include all four. In some particular instances, the composition will be formulated so that a typical unit dose thereof will provide approximately 400 to 300 milligrams of each component to a patient.

In this disclosure, all compositions, unless otherwise noted, will be given on a weight/weight basis. In one group of compositions, the green tea polyphenol component comprises 20-40%, the resveratrol comprises 5-20%, the soy isoflavones comprise 3-15%, and the curcumin comprises 30-70% of the composition. In a particular example of this group of compositions, the green tea polyphenol comprises 28%, the resveratrol comprises 11%, the soy isoflavones comprise 7%, and the curcumin comprises 54% of the material.

Another group of compositions comprises 40-70% of green tea polyphenols, 15-30% of resveratrol, 10-20% of soy isoflavones, and 2-15% of the curcumin. A specific example of this group of compositions comprises 56% green tea polyphenols, 22% resveratrol, 15% soy isoflavones and 7% curcumin.

The foregoing compositions and ranges have been found to provide a good synergistic interaction. However, other compositional ranges will be apparent and may be determined by those of skill in the art in view of the teaching presented herein.

The compositions may be readily administered on an oral basis in the form of capsules, tablets, gels, elixirs, or the like. In a typical treatment regimen, the composition may be administered to a patient over a fairly wide dosage range. Typical dosages will comprise at least 0.5 gram per day, and due to the low toxicity of the materials may range upward to 10 grams or more. A particular dosage range having utility for a broad range of healthy individuals as well as patients comprises 1-5 grams per day taken either singly or in a number of discrete doses. While the foregoing has described the administration of the material by oral dosing, it is to be understood that other dosing methods known in the art may be likewise employed. For example, the materials may be used in liquid extract form which may be administered intravenously. In other instances, the composition may be administered by suppositories, transdermally or by other methods known in the art. Further, the composition can be packaged in a unitary dose form, each unitary dose containing, on an independent basis, 40 to 300 milligrams of each of said components.

As mentioned above, the compositions of the present invention may be utilized as an adjunct to other methods for the promotion of wellness, the prevention of diseases or medical therapies for existing diseases including, but not limited to, chemotherapies, surgery, radiation, thermal therapies and the like. The composition of the present invention will have a primary action to prevent or control tissue inflammation and reduce pathophysiological events or promote healing by, among other things, inhibiting biochemical pathways involving the NF kappa B pathway. In addition, the composition will act to inhibit or moderate inflammation or other tissue damage resultant from diet, lifestyle, environmental influences or, in the case of treatment of disease, the therapy it is used in combination with.

Because of its action in preventing, moderating and inhibiting tissue damage, the composition of the present invention will have utility in treatment of trauma resultant from accidents and the like. The composition will also have utility in the treatment of tissue damage resultant from exposure to environmental factors such as sunlight, pollutants, and the like. The composition will also have utility moderating cumulative tissue damage associated with normal aging processes. As such, the composition will be seen to have utility in both the treatment and control of disease as well as in the general maintenance of health and wellbeing.

It is to be understood that the foregoing discussion, description and examples are illustrative of specific embodiments and implementations of the present invention. Still other embodiments and implementations will be apparent to those of skill in the art in view of the teaching presented herein. Accordingly, nothing in this description is meant to limit the practice of the invention. It is the following claims, including all equivalents, which define the scope of the invention.

What is claimed is:

1. A composition for inhibiting the activity of NF Kappa B comprising a combination of:
green tea polyphenol;
resveratrol;
soy isoflavone; and
curcumin.

2. The composition of claim 1, wherein the green tea polyphenol comprises a mixture of polyphenolic compounds extracted from tea leaves.

3. The composition of claim 1, comprising:
20-40% by weight polyphenol;
5-20% by weight resveratrol;
3-15% by weight soy isoflavones; and
30-70% by weight curcumin.

4. The composition of claim 1, comprising:
28% by weight polyphenol;
11% by weight resveratrol;
7% by weight soy isoflavones; and
54% by weight curcumin.

5. The composition of claim 1, comprising:
40-70% by weight polyphenol;
15-30% by weight resveratrol;
10-20% by weight soy isoflavones; and
2-15% by weight curcumin

6. The composition of claim 1, comprising:
56% by weight polyphenol;
22% by weight resveratrol;
15% by weight soy isoflavones; and
7% by weight curcumin.

7. A process of inhibiting the activity of NF kappa B comprising the step of orally administering the composition of claim 1 in the form of a capsule, tablet, elixir or gel.

8. The process of inhibiting the activity of NF kappa B as claimed in claim 7, wherein said composition comprises the form containing a does of from 40-300 mg.

9. The process of inhibiting the activity of NF kappa B as claimed in claim 8, wherein a daily dosage comprises 1-5 grams of the composition.

10. A process for decreasing oxidative or inflammatory damage to tissues comprising the step of orally administering the composition of claim 1 in the form of a capsule, tablet, elixir or gel.

11. The process of inhibiting the activity of NF kappa B as claimed in claim 10 wherein said composition comprises the form containing a does of from 40-300 mg.

12. The process of inhibiting the activity of NF kappa B as claimed in claim 11, wherein a daily dosage comprises 1-5 grams of the composition.

13. The process of claim 7, wherein a daily dose is at least 0.5 grams per day.

14. The process of claim 10, wherein a daily does is at least 0.5 grams per day.

15. A composition for inhibiting tissue damage and inflammation comprising:
green tea polyphenol;
resveratrol;
soy isoflavone; and
curcumin.

16. The composition of claim 15, wherein the green tea polyphenol comprises a mixture of polyphenolic compounds extracted from tea leaves.

17. The composition of claim 15, comprising:
20-40% by weight polyphenol;
5-20% by weight resveratrol;
3-15% by weight soy isoflavones; and
30-70% by weight curcumin.

18. The composition of claim 15, comprising:
28% by weight polyphenol;
11% by weight resveratrol;
7% by weight soy isoflavones; and
54% by weight curcumin.

19. The composition of claim 15, comprising:
40-70% by weight polyphenol;
15-30% by weight resveratrol;
10-20% by weight soy isoflavones; and
2-15% by weight curcumin

20. The composition of claim 15, comprising:
56% by weight polyphenol;
22% by weight resveratrol;
15% by weight soy isoflavones; and
7% by weight curcumin.
